(19) United States
(12) Patent Application Publication
(10) Pub. No.: US 2010/0203141 A1
(43) Pub. Date: Aug. 12, 2010

(54) STIMULI-RESPONSIVE LOW SOLUBILITY HYDROGEL COPOLYMERS OF N-ISOPROPYL ACRYLAMIDE AND SYNTHESIS METHOD

(75) Inventors: Alexander L. Yarin, Clarendon Hills, IL (US); Yiyun Zhang, Chicago, IL (US)

Correspondence Address: GREER, BURNS & CRAIN, 300 S WACKER DR, 25TH FLOOR, CHICAGO, IL, 60606, US

(73) Assignee: The Board of Trustees of the University of Illinois, Urbana, IL (US)

(21) Appl. No.: 12/703,266

(22) Filed: Feb. 10, 2010

Related U.S. Application Data
(60) Provisional application No. 61/151,557, filed on Feb. 11, 2009.
(60) Provisional application No. 61/163,216, filed on Mar. 25, 2009.

(51) Int. Cl. A61K 9/00; C08F 220/70; C08F 220/10; C08F 220/06; A61K 47/32; A61P 43/00; B29C 47/00

(52) U.S. Cl. 424/486; 526/303.1; 526/307.7; 526/307.6; 526/307.8; 514/772.4; 264/465

(57) ABSTRACT

Preferred embodiment stimuli-responsive low solubility copolymer hydrogel compositions of the invention include polymerized N-isopropylacrylamide and water insoluble monomer or oligomer repeating units. The repeating units are arranged within the polymer backbone of the copolymer hydrogel. In a preferred fabrication method, precursors of N-isopropylacrylamide and precursors of the water insoluble monomer of oligomer are solved in a liquid solvent to form a solution in a container. An initiator is added to the solution. Gas is bubbled through the solution, and the container is sealed. The solution is stirred while heating to a polymerization temperature and polymerization is permitted to complete to form the copolymer hydrogel composition.

### CLAIM FOR PRIORITY AND REFERENCE TO RELATED APPLICATION

This application claims priority pursuant to 35 U.S.C. §119 and all applicable statutes and treaties from prior provisional application Ser. No. 61/151,557 entitled Stimuli-Responsive Copolymers of N-Isopropyl Acrylamide Having Enhanced Longevity in Water, which was filed on Feb. 11, 2009, and from prior provisional application Ser. No. 61/163,216, entitled Stimuli-Responsive Copolymers of N-Isopropyl Acrylamide Having Enhanced Longevity in Water, which was filed on Mar. 25, 2009.

### STATEMENT OF GOVERNMENT INTEREST

This invention was made with government support under National Science Foundation Grant NIRT CBET-0609062. The government has certain rights in the invention.

### FIELD

A field of the invention is copolymer hydrogels. Another field of the invention is stimuli-responsive delivery vehicles for chemical substances that affect biological processes, sometimes referred to as nanobots. Another field of the invention is microfluidics. A microfluidics application of the invention is a capillary having an internal coating of a hydrogel copolymer of the invention to provide a stimuli responsive cross section. Copolymer coatings, both wettable and nonwettable, are another application of the invention. Another example application is to filter media, where a hydrogel copolymer of the invention can provide stimuli responsive release of trapped material. Additional applications will be apparent to artisans.

### BACKGROUND

Hydrogel copolymers can accept or expel water. Temperature sensitive shrinkage of hydrogel copolymers that expel water is an interesting property because it can be used to control the release of other materials. As an example, shrinkage of poly(N-isopropyl acrylamide) (PNIPAM) copolymers above lower critical solution temperature of 32.8° C. occurs because water entrapped in the polymer is released as a result of the increased attractive segmental interactions between the hydrophobic isopropyl groups of the PNIPAM chain. Such temperature sensitive copolymers have great potential in biomedical applications, such as vectors for drug delivery or in the purification of proteins and DNA. PNIPAM is an especially attractive candidate copolymer because its lower critical solution temperature of 32.8° C. is close to physiologic temperature. Practical use of PNIPAM is hindered, however, by very low longevity of its solid articles in water due to relatively rapid dissolution (on the scale of several hours to one day). Such high solubility is similarly impractical for any application that exposes the copolymer to water (including water vapor).

### SUMMARY OF THE INVENTION

Preferred embodiment stimuli-responsive low solubility copolymer hydrogel compositions of the invention include polymerized N-isopropylacrylamide and water insoluble monomer or oligomer repeating units. The repeating units are randomly arranged within the polymer backbone of the copolymer hydrogel. In a preferred fabrication method, precursors of N-isopropylacrylamide and precursors of the water insoluble monomer of oligomer are solved in a liquid solvent to form a solution in a container. An initiator is added to the solution. Gas is bubbled through the solution, and the container is sealed. The solution is stirred while heating to a polymerization temperature and polymerization is permitted to complete to form the copolymer hydrogel composition.

### BRIEF DESCRIPTION OF THE DRAWINGS

FIGS. 1-3 depict the measured DSC curves of experimental stimuli responsive low solubility P[(MMA/NIPAM)] copolymers CP 1-1, CP1-2 and CP1-3;

FIGS. 4 & 5 depict release experiments conducted with experimental responsive low solubility P[(MMA/NIPAM)] copolymer nanofiber mats of CP 1-1 subjected to temperature modulation in a water bath;

FIGS. 6 & 7 depict release experiments conducted with experimental responsive low solubility P[(MMA/NIPAM/AA)] copolymer nanofiber mats of CP2-3 subjected to pH modulation in a water bath;

FIGS. 8A and 8B illustrate coating thickness of P[(MMP/NIPAM]] within a glass micropaillary from experiments for pretreatment with HF and UV and AA and UV, respectively; and

FIG. 9 illustrates the results of pressure testing of copolymer coatings on microcapillary walls.

### LIST OF ACRONYMS

AA Acrylic acid
AIBN Azobisisobutyronitrile
MMA Methyl methacrylate
NIPAM N-isopropylacrylamide
PAA poly(acrylic acid)
PAN polyacrylonile
PMMA poly(methyl methacrylate)
PNIPAM poly(N-isopropyl acrylamide)
PS polystyrene
PVC polyvinyl chloride
RT room temperature
SCA static contact angle

### DETAILED DESCRIPTION OF THE PREFERRED EMBODIMENTS

The invention provides low solubility stimuli-responsive hydrogel copolymers that include an insoluble polymer randomly throughout the copolymer bulk material structure. Preferred embodiments of the invention provide stimuli-responsive low solubility hydrogel copolymers of NIPAM and methods for synthesis of the same. Preferred hydrogel NIPAM copolymers of the invention are relatively insoluble through the incorporation of a water insoluble monomer or oligomer structural unit that is randomly incorporated into the copolymer, which is provided as bulk material that can take the form of a wide variety of three dimensional shapes as well as thin coatings.

Copolymers of the invention have all units are randomly arranged within the polymer backbone, i.e., incorporated into the copolymer chain. In distinction from the traditional copolymers based on NIPAM, the copolymers of the present invention result in covalent bonds connecting all the monomers or oligomers, which enhances their stability in water. In the present copolymers have sufficiently high molecular weights to allow for easy processing, e.g. via electrospinning, which results in non-woven materials of interest for such applications as micro- and nanofluidics, disposable wipers, drug carriers, and tissue engineering. With lower solubility than prior NIPAM, a wide range of important applications are made practical. Preferred embodiment low solubility stimuli-responsive hydrogel copolymers of the invention are temperature sensitive. Additional preferred embodiment low solubility stimuli-responsive hydrogel copolymers of the invention are pH sensitive. Preferred applications of the invention include stimuli-responsive drug delivery devices (nanobots), fluid capillaries, coatings, and filter media. Preferred embodiment low solubility stimuli-responsive hydrogel copolymers of the invention can be synthesized by Free-Radical polymerization.

A preferred water insoluble monomer or oligomer structural unit is formed from the PMMA monomer or oligomer macromolecule. PMMA is biocompatible and preferred embodiment NIPAM-PMMA copolymers can be applied via electrospinning More generally, water insoluble monomer or oligomer structural units are produced from monomer or oligomer macromolecules that can be synthesized as a copolymer with NIPAM by Free-Radical polymerization, which requires the monomer or oligomer macromolecule to have an unpaired electron at its growing chain. Other suitable example water insoluble monomer or oligomer structural units can be formed from the PAN, PS, and PVC monomer or oligomer macromolecules. Appropriate initiator, solvent and reaction conditions are selected for copolymerization based upon the particular water insoluble monomer or oligomer macromolecule selected for copolymerization.

Additional preferred embodiment low solubility stimuli-responsive hydrogel copolymers include ionizable groups, such as carboxyl groups or amino groups. The inclusion of the ionizable groups makes the hydrogel copolymers responsive to pH changes. Ionizable groups are provided by a pH-sensitive monomer or oligomer macromolecule. Example monomer or oligomer macromolecules that can be synthesized as a copolymer with low solubility NIPAM of the invention include PAA, PEMA, and PMAA.

Preferred embodiments of the invention will now be discussed with respect to the drawings and experiments that have been conducted. Artisans will recognize broader aspects of the invention from the discussion of the experiments. While experiments were conducted with MMA as a water insoluble structural unit, artisans will appreciated that other water insoluble monomer and oligomer structural units can be used.

Experiments were conducted to provide a copolymer of NIPAM and MMA, which possesses a high molecular weight and has low solubility as a result of the water insoluble MMA structural unit. The copolymer retains the stimuli-responsive properties of NIPAM, specifically a volume phase transition (VPT) above lower critical solution temperature comparable to biological temperature of about 32.8° C. Additional experiments provided pH sensitivity by the addition of AA. AA molecules adjust their conformation due to the protonation-deprotonation equilibrium in aqueous solutions. The experimental copolymers having MMA, NIPAM and AA provided low solubility smart thermo- and pH-responsive materials. P(MMA/NIPAM) and P(MMA/NIPAM/AA) copolymers possess an enhanced longevity in aqueous surroundings compared to NIPAM.

An embodiment of this invention provides radical copolymerization of stimuli-responsive repeating units including poly(methyl methacrylate/N-isopropyl acrylamide) P(MMA/NIPAM) and poly(methyl methacrylate/N-isopropyl acrylamide/acrylic acid) P(MMA/NIPAM/AA) copolymers, which possess an enhanced longevity in aqueous surroundings. P(MMA/NIPAM) copolymers are thermo-responsive, whereas P(MMA/NIPAM/AA) copolymers are thermo- and pH-responsive. Such copolymers of NIPAM and MMA or NIPAM, MMA and AA are synthesized as random polymers, where all units are randomly arranged along the polymer backbone, or as comb-like polymers.

An embodiment of the invention is a delivery vehicle for a substance, such as a nanobot for drug delivery. The delivery vehicle includes a low solubility copolymer of the invention that is loaded with the substance to be delivered.

A method of delivering a substance of the invention introduces the delivery vehicle into an environment that exposes it to stimuli such as temperature or pH to break down the polymer and release the substance. In a preferred embodiment, a NIPAM-PMMA copolymer or NIPAM-PMMA-AA copolymer is prepared as a delivery vehicle and loaded with a substance. The delivery vehicle is placed into an environment, e.g., an animal host, where the temperature is greater than 23° C. An environment might also are alternatively include a pH level is less than about 8. The temperature or pH stimuli causes the delivery vehicle to gradually dissolve to delivery the substance. The gradual period for stimuli induced release of the substance will be a period of several days with the low solubility provided by preferred embodiment delivery vehicles of the invention. Experimental NIPAM-PMMA polymers soaked in water for four days only experienced a NIPAM loss in the range of 1-10%. Conventional NIPAM, on the other hand, dissolves in a very short time. The environment itself might not possess the required stimuli to cause dissolution of the delivery vehicle as an ambient condition. The delivery vehicle could respond in that case to the intentional addition of stimuli or to an elevated stimuli temperature or pH condition in the environment.

A preferred method of incorporating the substance to be delivered by the delivery vehicle is to dissolve or suspend the substance in a precursor of the copolymer delivery vehicle and embedding it into the copolymer during the process polymerization. In a preferred method, where a low solubility NIPAM based copolymer of the invention is formed into a drug delivery vehicle, a drug is dissolved in an appropriate solvent together with one of the precursors of the copolymer to be formed. After the solution is polymerized it can be electrospun, and a drug will be embedded in the solidified copolymer nanofibers. Alternatively, a drug can be added to copolymer precursor solution in the form of powder (with nanoscale particles). When the solution with the suspended drug is incorporated into the copolymer such as by electrospinning, the drug nanoparticles are embedded in the solidified copolymer nanofibers.

Low solubility NIPAM based copolymers of the invention were formed an tested in experiments. The experiments will now be discussed. Artisans will recognize additional features of preferred embodiments and broader aspects of the invention from the experiments.

### Experimental Synthesis & Characterization of NIPAM/MMA & NIPAM/MMA/AA

Materials: N-isopropyl acrylamide (NIPAM), methyl methacrylate (MMA), acrylic acid (AA), 2-methylpropionitrile (AIBN), Acetone, Ethanol, n-hexane, tetrahydrofuran (THF), and dimethyl formamide (DMF) were purchased from Sigma-Aldrich and used without further purification.

Preparation/Characterization of (MMA/NIPAM) co-polymers: Poly(methyl methacrylate/N-isopropyl acrylamide) [P(MMA/NIPAM)] copolymers were prepared via radical copolymerization. MMA and NIPAM monomers in a chosen mol:mol ratio were solved in ethanol, and AIBN was added as the initiator. Preferred embodiment NIPAM:MMA ratios are in the range of 5:5 to 7:3. The data provided below provides guidance for providing copolymers having other molar ratios and for determining ratios experimentally to provide desired solubility or stimuli response. Nitrogen was bubbled through the solution in a bottle for 5 minutes. After that, the bottle was tightly sealed. The solution in the sealed bottle was stirred by a magnetic stirrer at 300 rpm and heated to 60° C. Polymerization of the NIPAM:MMA according to the invention will proceed in a range of about 55-85° C., but the preferred range is 60-70° C. The co-polymerization reaction proceeded for 20 hours. Then, the resulting solution was opened and heated to evaporate most of the Ethanol. After that, the 10 mL residual material was added drop by drop to 100 mL of hexane, which was being stirred all the time for about 10 minutes. As a result, the [P(MMA/NIPAM)] copolymers had precipitated. After removal of hexane with the residual monomers, the copolymer sediments were washed by using ultrasound in hexane for 20 minutes. Finally the copolymer sediments were dried.

Preparation/Characterization of P(MMA/NIPAM/AA) co-polymers: Poly(methyl methacrylate/N-isopropyl acrylamide/acrylic acid) [P(MMA/NIPAM/AA)] copolymers were prepared via radical copolymerization, according to the following procedure. MMA, NIPAM and AA monomers in a chosen mol:mol ratio were solved in ethanol, and AIBN was added as the initiator. Preferred embodiment NIPAM:MMA:AA ratios are in the range of 5:10:5 to 3:10:7. The data provided below provides guidance for providing copolymers having other molar ratios and for determining ratios experimentally to provide desired solubility or stimuli response. The following procedure closely resembled the one described for copolymerization of P(MMA/NIPAM) copolymers.

Electrospinning: Poly(methyl methacrylate/N-isopropyl acrylamide) copolymers were dissolved in a mixture of dimethyl formamide (DMF) and acetone (2:8, v/v). For P(MMA/NIPAM/AA) copolymers, a blend of DMF, THF and ethanol (1:1:3, v/v/v) was used as a solvent. The solutions were electrospun through a nozzle. The concentrations, applied voltages, inter-electrode distance, flow rate and needle diameter are shown in Table 3. The nanofibers were collected as a partially oriented strip on a rotating grounded vertical metal disk. The rotation speed of the disk was 5000 rpm.

Leaching experiment: Electrospun strips of P(MMA/NIPAM) and P(MMA/NIPAM/AA) copolymers were weighed and then immersed in water for four days. After that, they were dried and weighed once again. The relative mass losses for the three P(MMA/NIPAM) copolymers are shown in Table 1. The corresponding data for P(MMA/NIPAM/AA) copolymers—in Table 2.

Dye release: The dye release experiments were carried out by cutting the fiber mats into small 1 mg pieces and immersing them in 2 mL of water in a glass vial. With a certain periodicity, water with the released dye was removed from the vial and replenished with the same amount of fresh water with a proper temperature for the temperature modulation. In the experiments with pH modulation, the aqueous solution with the released dye was removed and replenished with an aqueous solution of nitric acid with an appropriate pH value. The fluorescence of the removed supernatant was measured by a SpectraMax spectrofluorometer. The excitation and emission wavelengths were 540 nm and 610 nm respectively. The measured fluorescence levels were compared with a calibration curve and thus the amount of dye released from the nanofiber mats into water was found. To minimize the experimental error, 5 pieces of mats cut from the same electrospun nanofiber mat were used in each experiment and for each sample, and three separate fluorescence measurements were done simultaneously. The average value was used to minimize the error of the experiment.

Swelling/Shrinkage Transition: The swelling/shrinkage transition of NIPAM-based copolymers at LCST could be investigated by several experimental techniques, including static and dynamic laser light scattering, fluorescence emission, calorimetry, and visual observation of articles. The latter technique was the most appropriate in the experiments described here. Two additional techniques were employed to characterize the properties of the copolymers synthesized. In the first one, Differential Scanning Calorimetry was used to measure the glass transition temperature, T_{g} of copolymer samples and to delineate thermally-triggered reversible shrinkage from an irreversible creep-like shrinkage at higher temperatures. Differential Scanning Calorimetry was conducted using DSC Q200, TA instruments. The samples were heated from room temperature up to 160° C. at the heating rate of 10° C./min, using nitrogen as a purge gas at the flow rate of 100 mL/min. For each copolymer measurements were done for a bulk sample, as well as for the corresponding electrospun nanofiber mat.

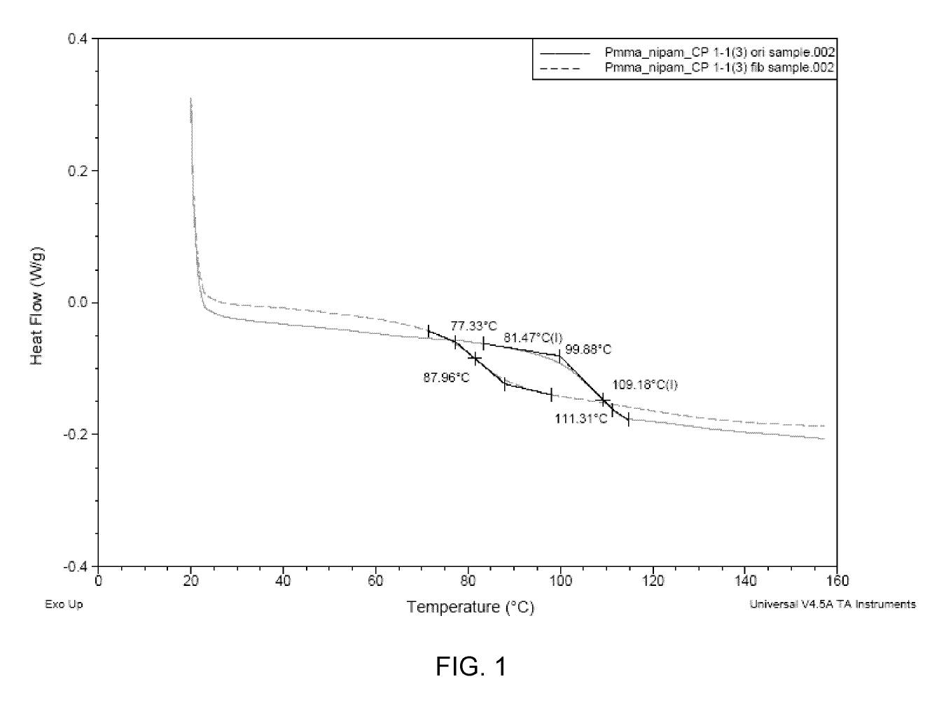

Wettability: The second additional characterization dealt with wettability by water at temperatures below and above LCST. High resolution, digitalized photo images were used to measure contact angles of water drops on the electrospun copolymer nanofiber mats at different temperatures (below and above LCST). Three different P(MMA/NIPAM) copolymers with different ratios of NIPAM to MMA were polymerized (cf. Table 1). Solutions of these copolymers were electrospun (under the conditions listed in Table 3) onto a rotating vertical grounded wheel with a 1 cm-wide strip over its circumference used as a counter-electrode. The resulting electrospun partially oriented nanofiber strips of these copolymers were used for their characterization. Samples of all three P(MMA/NIPAM) copolymers denoted CP1-1, CP1-2 and CP1-3 were cut from the electrospun strips and placed at the free surface of a double distilled water layer in a Petri dish. The samples floated, each one in its own compartment of the same dish, and were observed and photographed from above (FIG. 1).

The Petri dish was placed on a hot plate covered for the image contrast by black paper. Water temperature was gradually increased and simultaneously measured by a thermometer. The CP1-1, CP1-2 and CP1-3 samples of Table 1 were also simultaneously photographed for the further analysis of their thermo-responsive shrinkage at temperatures (a) 23° C. (b) 38.1° C. (c) 45° C. (d) 52° C. (e) 58° C. The images revealed sample shrinkage as temperature increased. The images were processed to determine the corresponding LCST and shrinkage ratio for each copolymer separately.

The data in Table 1 show that sample 1 with 5:5 mol:mol ratio of NIPAM and MMA revealed the LCST of 52° C. at which it shrank dramatically, with the two-dimensional shrinkage ratio of 75.98%. The shrinkage ratio here is defined as [(S_{before}−S_{after})/S_{before}]·100%, where S_{before} and S_{after} are the surface areas of the fiber mat in water before and after being heated, at 23° C. and 52° C., respectively.

When NIPAM content in copolymer (samples 2 and 3) increases compared to that of sample 1, the copolymer LCST approaches its value for PNIPAM. In particular, the data in Table 1 show that samples 2 and 3 (NIPAM:MMA mol:mol ratios of 6:4 and 7:3, respectively) shrank at LCST of 38° C., much closer to the value of 32.8° C. for PNIPAM. The corresponding shrinkage ratios were 85.94% and 88.37% for samples 2 and 3, respectively. The trend is clear; the higher the NIPAM content in a copolymer, the higher is its shrinkage ratio. The MMA content also affects the LCST, which means that embodiments of the invention provide copolymers having a predetermined (selected in advance at a particular level) LCST.

When temperature was gradually reduced from 58° C. to 23° C. the samples, which were located at the free surface of water did not swell and recover their initial shape. It was assumed that in samples located at the free surface such a recovery would also lead to stretching the surface layer of water, which is counteracted by surface tension, the obstacle which the forces responsible for NIPAM-water interaction cannot overbear. This assumption also means that samples fully submerged in water should recover. The latter was verified experimentally. Copolymer samples similar samples 1-3 were submerged in water and stayed in the water bulk during subsequent heating and cooling. The samples demonstrated full or partial recovery, as illustrated by the data in Table 4.

TABLE 1
The list of three P[(MMA/NIPAM)] copolymers synthesized in
the experiments and their measured characteristics when floating
at the free surface.

| | | | NIPAM mass | |
|---|---|---|---|---|
| | NIPAM:MMA | | retained in 4 | |
| Sample | (mol:mol) | LCST (° C.) | days L₁ (%) | Shrinkage (%) |
| CP1-1 | 5:5 | 52 | 89.94 | 75.98 |
| CP1-2 | 6:4 | 38 | 94.52 | 85.94 |
| CP1-3 | 7:3 | 38 | 99.62 | 88.37 |

TABLE 2
The list of three [P(MMA/NIPAM/AA)] copolymers synthesized
in the experiments and their measured characteristics when
floating at the free surface.

| | | | NIPAM mass | |
|---|---|---|---|---|
| | NIPAM:MMA:AA | pH response | retained in 4 | Shrinkage |
| Sample | (mol:mol:mol) | threshold | days L₂ (%) | (%) |
| CP2-1 | 5:10:5 | 4.4 | 68.27 | 6.3 |
| CP2-2 | 4:10:6 | 5.6 | 65.88 | 21.6 |
| CP2-3 | 3:10:7 | 6.4 | 32.67 | 45.7 |

TABLE 3
Electrospinning conditions.

| | | | | Interelectrode | | Needle |
|---|---|---|---|---|---|---|
| | Solvent | Concn. | Voltage U | Distance | Flow rate | o.d. |
| Sample | (v:v) | (wt %) | (kV) | H (cm) | (mL/h) | (mm) |
| CP1-1 | DMF:Ace | 6.3 | 12.5 | 12 | 0.8 | 0.5 |
| | (2:8) | | | | | |
| CP1-2 | DMF:Ace | 19.0 | 13.5 | 12 | 0.8 | 0.5 |
| | (2:8) | | | | | |
| CP1-3 | DMF:Ace | 16.7 | 15.0 | 12 | 0.8 | 0.5 |
| | (2:8) | | | | | |
| CP2-1 | DMF:THF:EtOH | 15.4 | 20 | 12 | 1 | 0.5 |
| | (1:1:3) | | | | | |
| CP2-2 | DMF:THF:EtOH | 20.0 | 17.5 | 12 | 1 | 0.5 |
| | (1:1:3) | | | | | |
| CP2-3 | DMF:THF:EtOH | 18.6 | 20 | 12 | 2 | 0.5 |
| | (1:1:3) | | | | | |

TABLE 4
The list of three P[(MMA/NIPAM)] copolymers synthesized in
the experiments and their swelling/shrinkage characteristics when
fully submerged in water.

| | NIPAM:MMA | | | |
|---|---|---|---|---|
| Sample | (mol:mol) | LCST (° C.) | Shrinkage (%) | Swelling (%) |
| CP1-1 | 5:5 | 52 | 35.56 | 30.05 |
| CP1-2 | 6:4 | 38 | 73.21 | 39.33 |
| CP1-3 | 7:3 | 38 | 71.76 | 35.91 |

Presence of the acrylic acid (AA) monomers in a copolymer adds pH-responsive properties. Three different P(MMA/NIPAM/AA) copolymers with different ratios of NIPAM, MMA and AA were polymerized (Table 2). Solutions of these copolymers were electrospun (under the conditions listed in Table 3) similarly to the P(MMA/NIPAM) copolymers CP2-1, CP2-2 and CP2-3 were placed at the free surface of a double distilled water layer in a Petri dish.

Water pH was gradually changed by adding diluted nitric acid, drop by drop, and simultaneously measured by the Oakton pH-meter, pHtestr 30. The samples were also simultaneously photographed for the further analysis of their pH-responsive shrinkage. A succession of images was taken over a time span of about 40 min. The images clearly showed sample shrinkage as pH decreased. The images were processed to determine the corresponding pH-threshold for shrinkage and the corresponding shrinkage ratio for each copolymer separately. The data in Table 2 show that sample 3 (CP2-3) with 3:10:7 mol ratio of NIPAM, MMA and AA revealed a pH-threshold for shrinkage of 6.4, at which it shrank dramatically, with the two-dimensional shrinkage ratio of about 45.7%. The shrinkage ratio here is defined as [(S_{before}−S_{after})/S_{before}]·100%, where S_{before} and S_{after} are the surface areas of the fiber mat in water before and after changing the water pH to the value corresponding to the pH-threshold for shrinkage for each sample.

When the AA content in copolymer (CP2-2 and CP2-3) increases compared to that of CP2-1, the corresponding pH-threshold for shrinkage increases as well. This allows selection of a predetermined pH-threshold by selection of an appropriate AA content. In particular, the data in Table 2 show that samples 1 and 2 (NIPAM:MMA:AA mol ratios of 5:10:5 and 4:10:6, respectively) shrank at pH values of 4.4 and 5.6, respectively. The corresponding two-dimensional shrinkage ratios were around 6.3% and 21.6%, respectively.

The ability to set a response for shrinkage of the copolymer at a predetermined pH provides an important feature for many applications. As an example, cancer tumors and inflamed tissues are typically more acidic (pH 6.5) than normal tissues (pH 7.4). Therefore, the pH-threshold of CP2-3 at a body temperature of about 37° C. allows for distinguishing between cancer tumors and normal tissues, which makes this copolymer a good candidate for smart delivery drug carriers capable of a highly localized drug release to diminish severe side effects of a number of anti-cancer therapeutics.

In separate experiments, mass losses in water of the three P(MMA/NIPAM) copolymers of Table 1, and of the three P(MMA/NIPAM/AA) copolymers of Table 2 were evaluated. The samples were submerged in water for four days at room temperature. To process the data of these experiments, denote by μᵢ and r₁ molecular weights and mole fractions of all monomers involved in the original copolymers (with i=1 for NIPAM, i=2 for MMA, and i=3 for AA). Then, the effective molecular weight of a P(MMA/NIPAM) copolymer is μₑ₁=μ₁r₁+μ₂r₂, and that of a P(MMA/NIPAM/AA) copolymer is μₑ₂=μ₁r₁+μ₂r₂+μ₃r₃. Let the initial sample mass be M₀, which means that it contains M₀/μₑ₁ moles of a P(MMA/NIPAM) copolymer, or M₀/μₑ₂ moles of a P(MMA/NIPAM/AA) copolymer. Then, the corresponding number of NIPAM moles in these samples is r₁M₀/μₑ₁ and r₁M₀/μₑ₂, respectively. Let the total mass loss of a copolymer in four days in water be ΔM. Assuming that in the case of the P(MMA/NIPAM) copolymers only NIPAM is lost (since MMA is water insoluble), the number of the lost NIPAM moles is ΔM/μ₁, whereas the relative remaining mass of NIPAM L₁ is found as μ₁ (r₁M₀/μₑ₁−ΔM/μ₁)/(μ₁r₁M₀/μₑ₁), i.e. is given by the following formula

$${L}_{1} = [ 1 - \frac{Δ M}{{M}_{0}} \frac{( {μ}_{1} {r}_{1} + {μ}_{2} {r}_{2} )}{{μ}_{1} {r}_{1}} ] \cdot 100 \% \qquad ( 1 )$$

In the case of the P(MMA/NIPAM/AA) copolymers, not only NIPAM but also AA is water soluble. Therefore, assuming that the mass losses are due to both NIPAM and AA (but not due to the water insoluble MMA), the effective molecular weight of the NIPAM/AA losses is μₗₒₛₛ=(μ₁r₁+μ₃r₃)/(r₁+r₂). Then, the number of lost NIPAM moles is given by ΔMr₁/[μₗₒₛₛ(r₁+r₃)] and thus the relative remaining mass of NIPAM L₂ is found as μ₁[r₁M₀/μₑ₂−ΔA Mr₁/[μₗₒₛₛ(r₁+r₃)]]/(μ₁r₁M₀/μₑ₂), i.e. is given by the following formula

$${L}_{2} = [ 1 - \frac{Δ M}{{M}_{0}} \frac{( {μ}_{1} {r}_{1} + {μ}_{2} {r}_{2} + {μ}_{3} {r}_{3} )}{( {μ}_{1} {r}_{1} + {μ}_{3} {r}_{3} )} ] \cdot 100 \% \qquad ( 2 )$$

Eqs. (1) and (2) include the assumption that insoluble MMA does not comprise any part of the mass loss. When mass losses are low, it is plausible that only individual water soluble monomers (NIPAM or AA) disappear from the copolymers, while the whole macromolecule is still intact. On the other hand, when sufficiently high mass losses occur, it is almost inevitable that some MMA appendices are lost with the NIPAM and/or AA monomers, even though MMA itself is water insoluble. Then, the estimates based on Eqs. (1) and (2) will be inaccurate.

In the calculations based on Eqs. (1) and (2), the following values of the parameters were used: μ₁=113.1 Da, μ₂=100.12 Da, μ₃=72.06 Da, and also: for CP1-1 r₁=0.5, r₂=0.5; for CP1-2 r₁=0.6, r₂=0.4; for CP1-3 r₁=0.7, r₂=0.3; for CP2-1 r₁=0.25, r₂=0.5, r₃=0.25; for CP2-2 r₁=0.2, r₂=0.3, r₃=0.5; for CP2-3 r₁=0.15, r₂=0.35, r₃=0.5.

The mass loss findings for the P(MMA/NIPAM) copolymers reveal trends similar to those found for their LCST. In particular, for sample 1, with lower initial NIPAM content, the NIPAM mass left estimated via Eq. (1) was 89.94% in four days (Table 1). For the samples 2 and 3 with a higher initial NIPAM content in Table 1, mass losses of NIPAM are higher and most probably lead to losses of MMA appendices as well. This makes Eq. (1) inapplicable. Since NIPAM losses from sample 1 are the lowest, the corresponding P(MMA/NIPAM) copolymer was chosen as a preferable material for the release experiments described below.

For P(MMA/NIPAM/AA) copolymers with the identical initial MMA content in samples CP2-1, CP2-2 and CP2-3, the NIPAM masses left estimated via Eq. (2) were 68.27%, 65.88% and 32.67% in four days (Table 2). Since NIPAM losses from sample CP2-3 are the lowest, and the initial AA content in sample CP2-3 is the highest, copolymer CP2-3 was chosen as a preferable material for the release experiments described below.

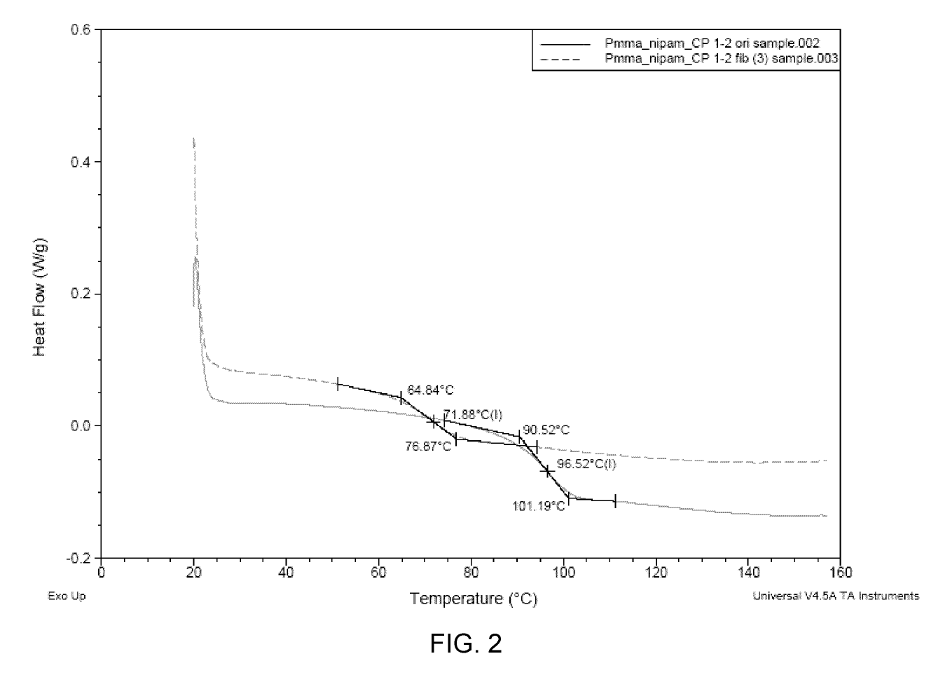

FIGS. 1-3 depict the measured DSC curves of the P[(MMA/NIPAM)] copolymers CP 1-1, CP1-2 and CP1-3, respectively. For each copolymer measurements were done for a bulk sample, as well as for the corresponding electrospun nanofiber mat. The results show that the electrospun mats have a reduced glass transition temperature compared to the one of the corresponding bulk samples. This phenomenon is attributed to fast solidification characteristic to electrospinning (where solvent evaporates from nanofiber surfaces with very high curvatures) compared to solidification of bulk macroscopic samples produced from solution puddles left drying in open air. The DSC results shown in FIGS. 2-4 clearly demonstrate that at temperatures up to about 70° C. copolymers CP1-1, CP1-2 and CP1-3 do not undergo glass transition. Therefore, their shrinkage at much lower temperatures as in Tables 1 and 4 is not related to glass transition and high-temperature irreversible creep-like shrinkage.

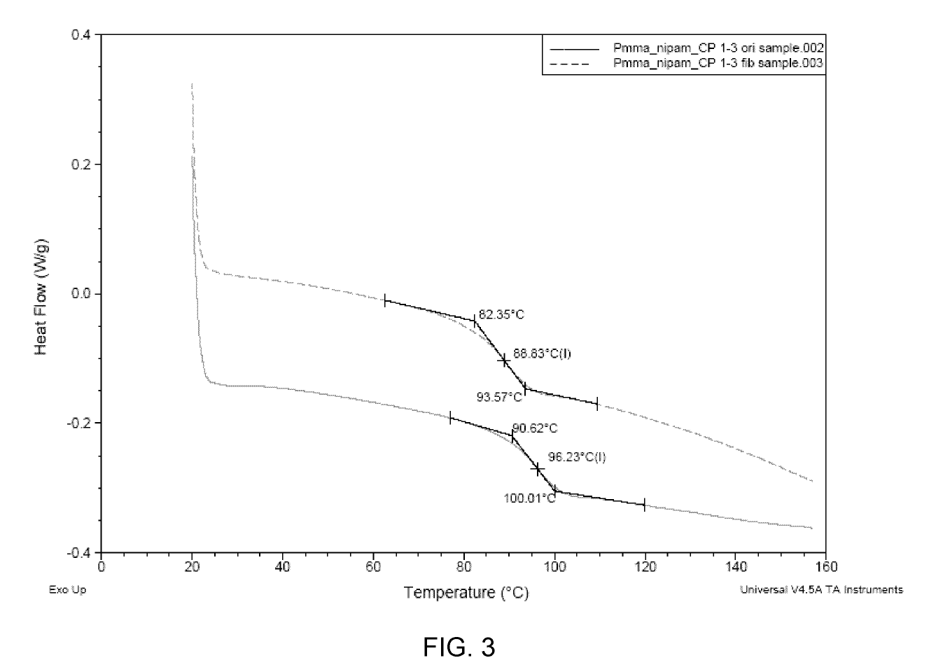

In FIG. 1, the DSC curve of the P[(MMA/NIPAM)] copolymer CP1-1 shows the results for the electrospun nanofiber mat, whereas the solid line is for the bulk sample. The glass transition temperature T_{g} of the bulk copolymer CP1-1 is 109.18° C., and that of the corresponding electrospun nanofiber mat is 87.96° C. In FIG. 2, the DSC curve of the P[(MMA/NIPAM)] copolymer CP1-2 shows the results for the electrospun nanofiber mat, whereas the solid line is for the bulk sample. The glass transition temperature T_{g} of the bulk copolymer CP1-2 is 96.52° C., and that of the corresponding electrospun nanofiber mat is 71.88° C. In FIG. 3, the DSC curve of the P[(MMA/NIPAM)] copolymer CP1-3 shows the results for the electrospun nanofiber mat, whereas and the solid line is for the bulk sample. The glass transition temperature T_{g} of the bulk copolymer CP1-3 is 96.23° C., and that of the corresponding electrospun nanofiber mat is 88.83° C.

Table 5 presents the static contact angles θ (SCA) of the copolymer intact spin-coated films and electrospun nanofiber mats. The sample temperature was controlled by a hot plate underneath the samples. Temperature was changed from the room temperature (of around 20° C.) to a high temperature (of about 40-50° C., depending on the copolymer) and then back to the room temperature. In several cases, an additional increase of temperature was added. The liquid used for determining the contact angles was deionized water. Water drops were placed on the sample surface using a syringe. The contact angle reported in Table 5 was an average of at least four readings at different places on the same sample.

TABLE 5
Static contact angles (SCA) of water drops on copolymer films
and nanofiber mats. The room temperature (RT) was around
20° C. For CP1-1, CP1-2 and CP1-3 the high temperatures
were 53° C., 46° C. and 39° C., respectively, all
above their corresponding LCSTs.

| | | | | SCA θ |
|---|---|---|---|---|
| | | SCA θ at high | SCA θ | at high |
| | SCA θ at RT | Temperature | at RT | Temperature |
| Sample | (degree) | (degree) | (degree) | (degree) |
| CP1-1 film | 60.27 | 70.45 | 54.71 | 74.44 |
| CP1-2 film | 32.45 | 53.58 | 29.95 | 46.30 |
| CP1-3 film | 27.26 | 30.43 | 33.63 | — |
| CP1-1 mat | 116.43 (at t = 0 s) | 129.10 | 112.64 | — |
| CP1-2 mat | 110.27 (at t = 0 s) | 122.93 | 59.69 | — |
| CP1-3 mat | 104.43 (at t = 0 s) | 130.65 | 84.46 | — |

Sample images were taken and used to measure SCA. The images showed that at room temperature both the film and mat are hydrophilic and are wetted by water (in the case of the mat at room temperature it takes about 50 s until water fully displaces air from the surface layer pores). The film becomes less wettable at 46° C. (above LCST of 38° C.), but still possesses a relatively small contact angle of 53.58°. The variation of the contact angle with temperature is fully reversible. On the other hand, the electrospun mat at 46° C. becomes hydrophobic and acquires the contact angle of about 122.93° (an almost 100° increase compared to the mat at room temperature in 50 sec after the drop was deposited). The hydrophobicity of the mat can be reversibly eliminated by reducing temperature back to the room temperature, demonstrating clearly the temperature dependent hydrophobicity that is provided by the present copolymers.

### Experimental NIPAM/MMA Drug Delivery Testing

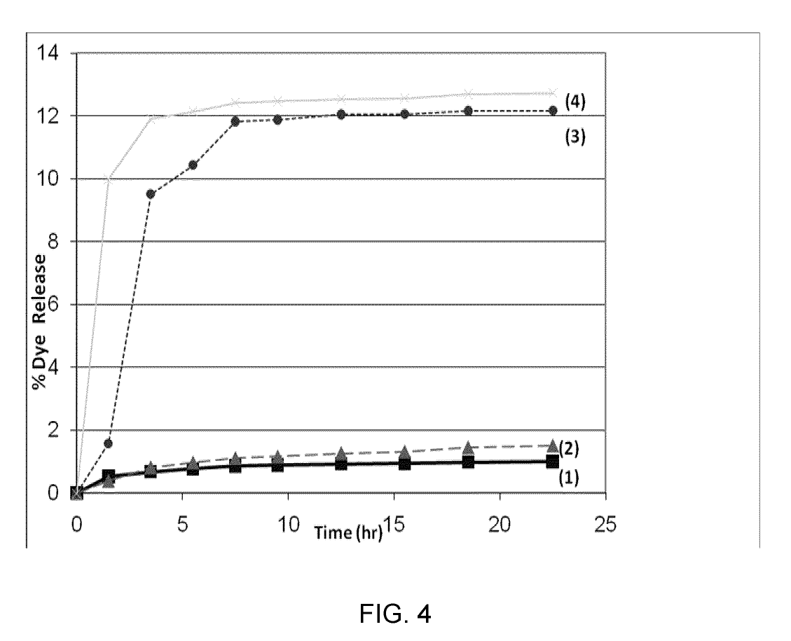

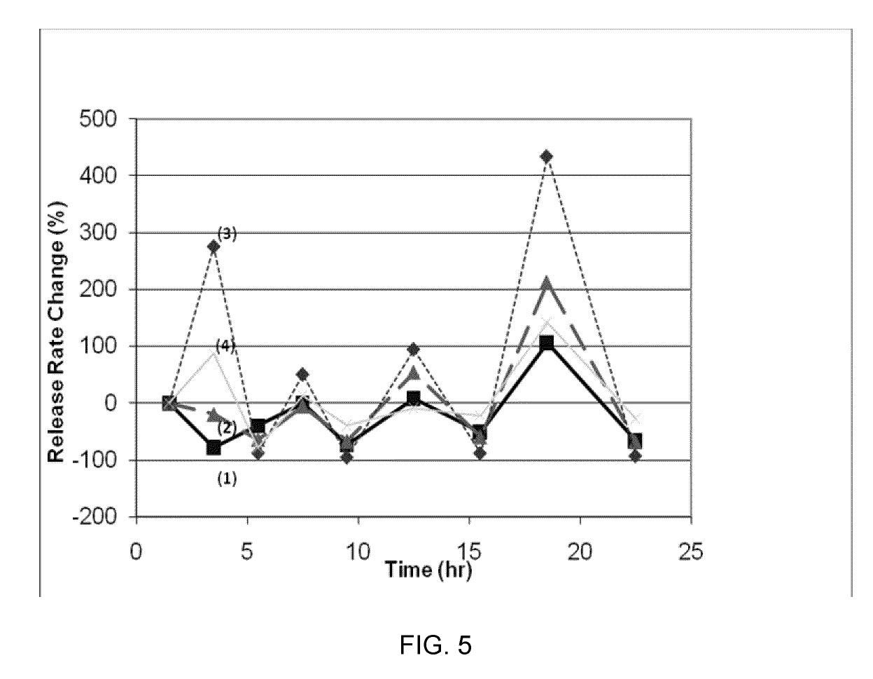

The ability of the present copolymers to act as a delivery vehicle was also tested. In the tests, fluorescent dye Rhodamine 610 chloride was incorporated into the electrospun nanofiber mats by dissolving it in copolymer solutions before electrospinning at concentration of 4.457·10⁻⁴ g/mL The fluorescent dye serves as a good model that is a convenient and useful agent capable of mimicking and elucidating of peculiarities of release of low and high molecular weight drugs. In the dye release experiments with P(MMA/NIPAM), sample nanofiber mat pieces were dipped in a water bath and the temperature was changed every 2 hours for the first 8 hours, then every 3 hours for the next 9 hours, and then, every 4 hours for the last 8 hours. Four series of the experiments were carried out along this scenario, with a temperature switch from 5 to 25° C. for series 1, from 25 to 45° C. for series 2, from 45 to 65° C. for series 3 and from 65 to 85° C. for series 4. The results are show in FIGS. 4 & 5. FIG. 4 plots the cumulative dye release versus time for P(MMA/NIPAM) copolymer mats (CP1-1) subjected to temperature modulation in a water bath. Curve 1- for series 1, curve 2- for series 2, curve 3- for series 3, and curve 4- for series 4. FIG. 5 plots the change of the release rate in the temperature modulation experiments of FIG. 4. Curve 1- for series 1, curve 2- for series 2, curve 3- for series 3, and curve 4- for series 4.

Both curves with temperature variation below the LCST of 52° C. (series 1 and 2) show a relatively low cumulative release of the order of 1% resembling that of pure PMMA nanofibers. The temperature sensitivity increases to 200% at 45° C. (close to the LCST). It is likely that the thermo-sensitivity of the release kinetics in series 1 and 2 in FIGS. 4 and 5 results from the thermal dependence of the dye desorption from nanopore surfaces, which is the rate-limiting process. It was still a rate-limiting process in the experiments, since all the release curves in FIG. 4 seemingly saturate well below 100%. In series 3, temperature crosses the LCST at 52° C. The corresponding cumulative release rapidly achieves the level of about 10% and saturates at about 12%. The release rate for series 3 and 4 have the same slope due to the same reason-crossing the LCST, as is seen in FIG. 4. For series 4, where temperature varies above the LCST, the cumulative release curve resembles that of series 3. This probably manifests the fact that the major release event happens at the preparatory stage of heating to 65° C. (the starting temperature of series 4) when the temperature inevitably crosses the LCST. The subsequent release kinetics corresponding to series 4 resembles that of series 1 and 2, i.e. most probably stems from the temperature dependence of the desorption process. The rate changes in series 4 in FIG. 4 do not reach the high levels characteristic of the release with crossing the LCST as in series 3. Artisans will appreciate that the copolymer P(MMA/NIPAM) CP1-1 exhibits a positively thermo-sensitive release profile (i.e. a higher release rate when it shrinks above LCST as compared to that below LCST when it swells), whereas release patterns of most other PNIPAM-based hydrogels known today are negatively thermo-sensitive. This remarkable behavior can be attributed to creation of new nanopores/nanocracks in CP1-1 when NIPAM nanogel islands shrink at temperatures higher than its LCST.

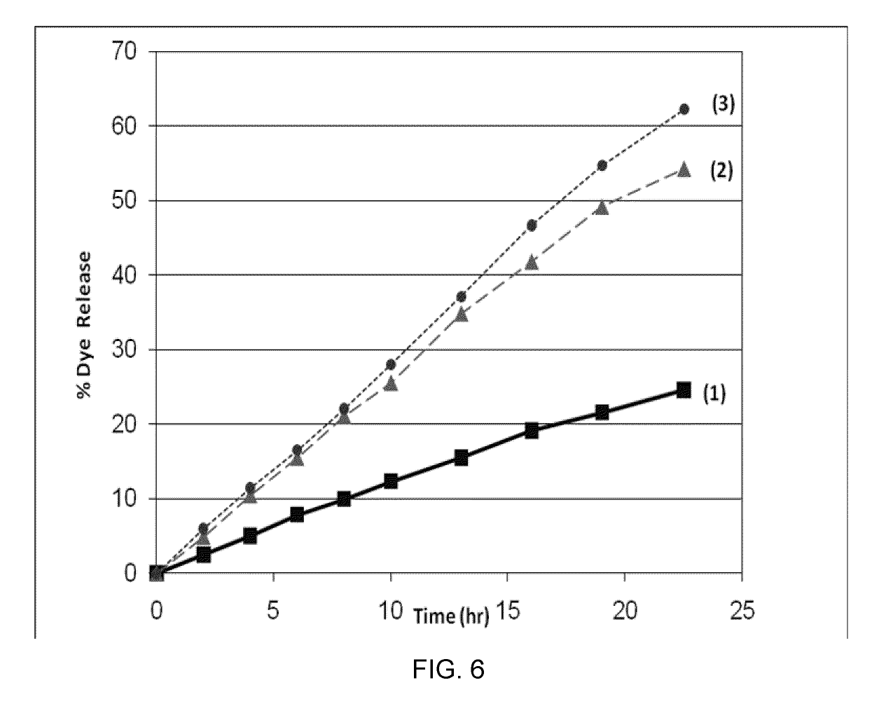

Experiments were also conducted to test pH sensitivity of copolymers of the invention, and the results are shown in FIGS. 6 and 7. FIG. 6 shows the cumulative dye release versus time for P(MMA/NIPAM/AA) copolymer CP2-3 mats subjected to pH modulation in the water bath. FIG. 7 shows the change of the release rate during the pH modulation experiments of FIG. 6. Curve 1- for series 1, curve 2- for series 2, and curve 3 - for series 3, Curve 1- for series 1, curve 2- for series 2, and curve 3- for series 3. During the experiments with pH modulation in a water bath containing P(MMA/NIPAM/AA) copolymer CP2-3, the temperature was sustained at 37° C. which is close to the human body temperature, whereas pH level was changed every 2 hours for the first 10 hours, then every 3 hours for the next 9 hours, and then, every 3.5 hours for the last 7 hours. There were three series of experiments with P(MMA/NIPAM/AA) following this scenario, with pH varied from 5 to 6 for series 1, from 6 to 7 for series 2, from 7 to 8 for series 3. With a certain periodicity, the aqueous solution with the released dye was removed from the vial and replenished with the same amount of aqueous solution of nitric acid with an appropriate pH value (5, 6, 7 or 8). The results of the experiments with dye release from P(MMA/NIPAM/AA) CP2-3 with pH modulation demonstrate that a drastic change in the cumulative release levels and the release rate between pH of 6 and 7. Such pH response, for example, makes the copolymer a suitable for drug carriers capable of distinguishing cancer tumors from normal tissues.

### Experimental NIPAM/MMA Microfluidic Flow Regulation Testing

The ability of present copolymers to provide stimuli responsive flow control in microcapillaries was also tested. The experiments demonstrated thermo-responsive on-demand regulation of water flow rate in glass microcapillaries through the present P(MMA/NIPAM) copolymer grafted at the inner walls of the microcapillaries. The experiments shoed that the P(MMA/NIPAM) copolymer coatings are stable and can withstand significant temperature variations. As the P(MMA/NIPAM) copolymers expand and contract in response to temperature, which changes the bore (flow cross section) of the capillary and therefore performs the function of on demand flow regulation. Changing temperature across LCST of the copolymer coating causes it to swell or shrink thus changing the bore available for pressure-driven flow. Selection of mol:mol ratios of the monomers and of the particular insoluble monomer used in the copolymer. Thus, the responsive temperature of the flow control can be selected within the range of available LCST of the copolymer of the invention that is used.

In the experiments, the grafted P(MMA/NIPAM) copolymer layers were subjected to different pressure drops applied to the capillary open ends, as well as to periodic temperature variation across the copolymer LCST to determine the best grafting conditions for microfluidic operation. By varying temperature, flow rate in the capillaries was changed periodically on demand due to the swelling/shrinkage of the grafted copolymer layer. Grafting conditions should avoid excess entrapment of air bubbles between the P(MMA/NIPAM) and the microcapillary wall. The coatings are stable, but testing showed that entrapped air bubbles in the coating can be responsible for apparent slippage with the slip lengths of the order of 7-10 μm. Some air bubbles are inevitably present in the fluffy copolymer grafted coating and create air voids. The water slip on the voids can be beneficial in certain applications, for example permitting a higher flow rate in reverse osmosis applications. Copolymer adhesion to microcapillary surfaces can also be improved by known methods for polymer-glass adhesion. Example techniques include modification of the glass chemistry, bombardment of the glass surface with nanoparticles, and UV exposure, which can provide some additional roughness and chemically active sites on the glass surface, both beneficial for an enhanced adhesion. Additionally, the present copolymers can be grafted to microcapillaries of other materials. Surface preparations that aid bonding will depend upon the material of the capillary.

Preparation of NIPAM/MMA Coated Capillaries

Pretreatment of glass microcapillaries: Two types of pretreatment for [P(MMA/NIPAM)] copolymer coating grafting to the inner walls of glass microcapillaries were tested. In the first one, roughening of glass surface with etching by hydrofluoric acid was followed by UV exposure to provide some additional active sites on the glass surface. Pretreatment of macroscopic glass microcapillaries (about 0.5 mm dia.) with hydrofluoric acid and UV exposure was conducted according to the following procedure. Three sets of glass microcapillaries were filled with 2% hydrofluoric acid due to wettability and were left for 2 min, 4 min, and 8 min. After that, hydrofluoric acid was evacuated from them by low-speed air flow, and then they were rinsed thoroughly with water. In addition, for comparison, one set of microcapillaries was filled with 2% hydrofluoric acid for 8 min and then treated by UV light for three hours. Additionally, for comparison, one set of microcapillaries was placed under UV light at a distance of approximately 40 cm from the light source for three hours. Also, for comparison, one set of glass microcapillaries was used without any pretreatment.

In the second type of pretreatment, after UV exposure for three hours, 5 mL of AA (acrylic acid) and 1 mL of H₂SO₄ were mixed together and used to fill microcapillaries. In this process the esterification reaction of carboxyl groups of AA with the hydroxyls on the capillary surface was used to bond AA monomers on the glass surface. Four hours later, the microcapillaries were rinsed by water completely.

Copolymerization and grafting: After a pretreatment, copolymerization and grafting of [P(MMA/NIPAM)] copolymer (one of the several copolymers introduced by the authors in Ref. 2, namely CP1-3) was conducted via the radical copolymerization described above. The pretreated with hydrofluoric acid and non-pretreated glass microcapillaries were cut and placed under UV light for an hour. After that, they were immersed in the copolymer solution for a 20 h period while being heated to 60° C., which facilitated grafting. Then, the microcapillaries were removed from the solution and heated until dried. The copolymer was grafted to both of the inside and outside of the microcapillaries. The outside grafting was removed prior to the thermal response testing by rinsing the outside of the capillaries in methanol.

Coating Pretreatment

The microcapillaries with [P(MMA/NIPAM)] copolymer coatings grafted at the inner surface after HF+UV pretreatment were installed in hypodermic needles and sealed with epoxy. After 24 h the epoxy was completely solidified and the needles with microcapillaries at the end were tested. The coatings produced by this method were insufficiently mechanically robust for pressure drops above 700-800 mbar.

To improve stability and adhesion of the coating, the second type of pretreatment was used, which results in glass microcapillaries with bonded AA monomers, which can be easily polymerized with MMA and NIPAM. An AA coating is formed on the capillaries. These pretreated glass microcapillaries with bonded AA monomers on the inner surface were submerged in the solution undergoing the copolymerization reaction at 60° C. for 20 h. Then, the glass microcapillaries were removed from the solution and heated until dried. The coatings were smooth and did not deteriorate when the microcapillaries were subjected to pressure-driven water flows at room and elevated (around 50° C.) temperatures.

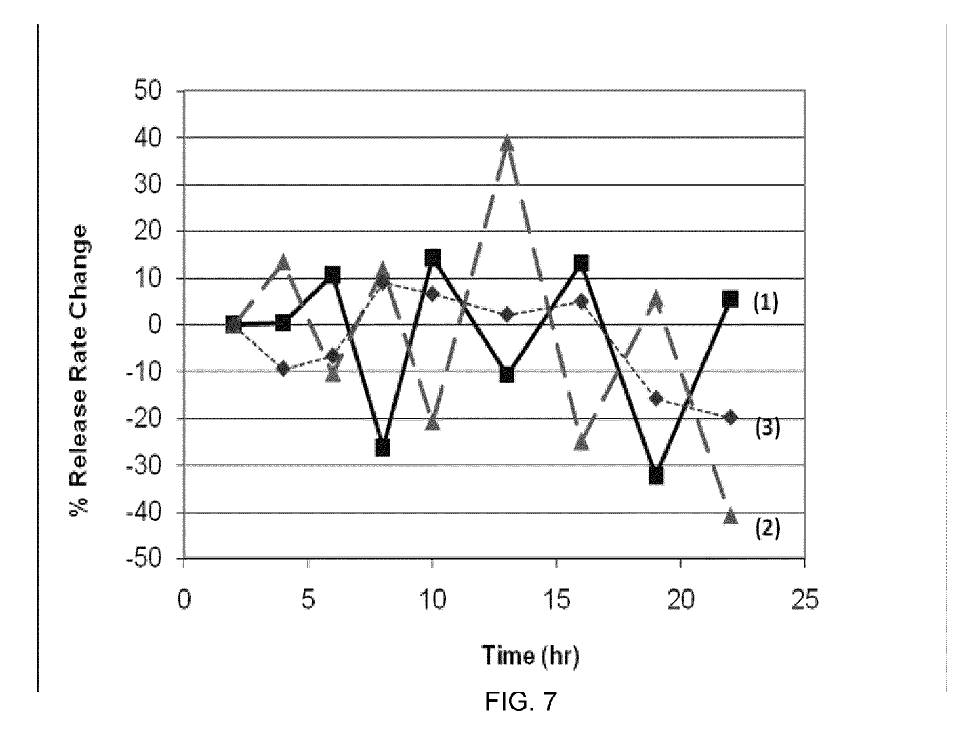

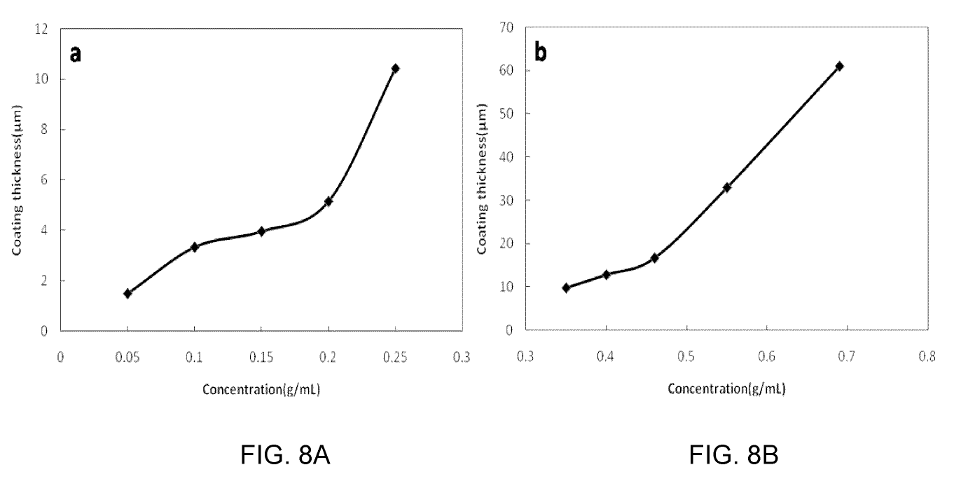

In the case of glass microcapillaries pretreated using HF etching and UV exposure, the concentration of solution used for copolymer grafting determined the coating thickness achieved in a certain time, and the results are shown in FIG. 8A. Thick coatings can swell to close a significant part or all of the microcapillary bore, and thinner coatings provide a lesser restriction. An optimal concentration HF and UV pretreatment was determined to be 0.2 g/mL as shown in FIG. 8A. The data for glass microcapillaries pretreated with AA and UV shows the optimal concentration was 0.55 g/mL as seen in FIG. 8B.

Mechanical Strength Pressure Testing

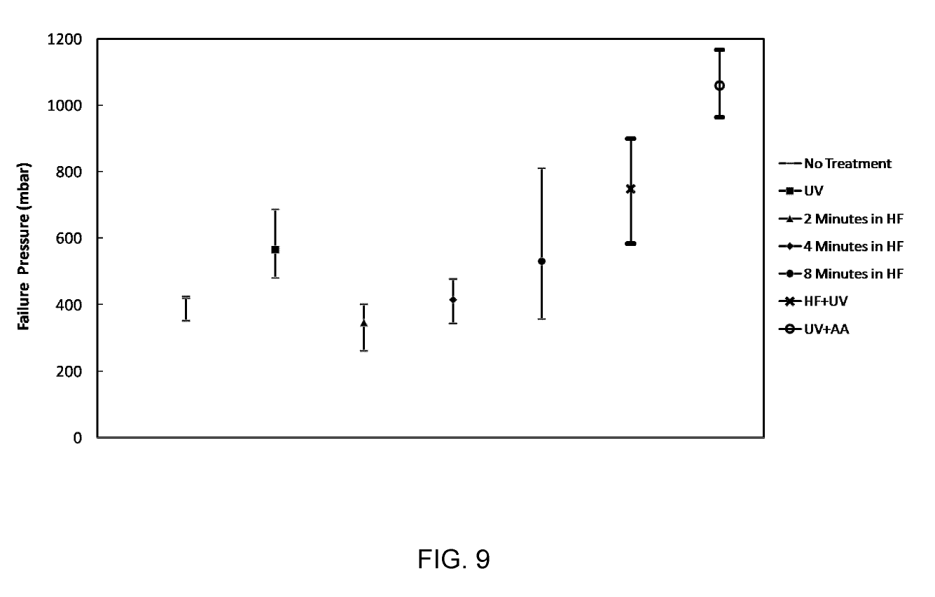

Pressure testing of the copolymer coatings on the inner microcapillary walls was conducted to evaluate their mechanical strength when subjected to pressure-driven water flow through the bore. In the experiment pressurized air in a syringe pushed water supplied from the syringe at room temperature through the glass capillary. The inner diameters of dry microcapillaries with the inner copolymer coatings were measured. Then, the microcapillaries were pre-wetted in water for 10 min. After that, the microcapillaries were subjected to increasing air pressure, which is recorded using an Omegalynx HPP-2023 pressure gauge. Pressure was measured every five seconds. Above a certain pressure level driving water flow, the copolymer coatings experienced mechanical failure, detached from the inner glass surface and slipped out of the capillary. The corresponding failure pressures are presented in FIG. 9.

The data show that the most mechanically robust coatings were grafted using the route involving the esterification of AA and UV pretreatment with the subsequent copolymer grafting. Coatings grafted after such pretreatment did not deteriorate when hot water at 48° C. was flowing through the microcapillaries. The mechanically robust samples were used for the thermo responsive flow control experiments.

Thermo-Responsive Flow Control

The results obtained in flows with temperature variation across LCST are shown in Tables 6 and 7. At room temperature of 20° C. (below LCST=38° C. of the copolymer CP1-3), the recorded flow rate was significantly smaller than the one at the elevated temperature of 45° C. This shows the copolymer layer swells in contact with water below LCST and obstructs a part of the bore, thus reducing the flow rate. At the temperature above LCST, the copolymer layer shrinks, the bore becomes wider and the volumetric flow rate increases. The effect of the bore radius increase is significantly amplified by the fact that volumetric flow rate is a strongly nonlinear function of ˜a⁴. The results showed that the present NIPAM/MMA copolymer layers of thicknesses of the order of 30 μm are a powerful tool for flow regulation in microcapillaries. Significantly, the results show that even after temperature crosses the LCST several times, the thermo-responsive control of volumetric water flow rate is maintained.

TABLE 6
Thermo-responsive flow through a copolymer-coated
microcapillary at lower pressure drop of 13.6 mbar

| Case | Temperature, ° C. | Q, cm³/s |
|---|---|---|
| 4 | Below LCST: 20° C. | 0.08345 |
| 5 | Above LCST: 45° C. | 0.17003 |
| 6 | Below LCST: 26° C. | 0.07006 |

TABLE 7
Thermo-responsive flow through a copolymer-coated
microcapillary at higher pressure drop of 23.4 mbar

| Case | Temperature, ° C. | Q, cm³/s |
|---|---|---|
| 7 | Below LCST: 27° C. | 0.1682 |
| 8 | Above LCST: 42° C. | 0.26626 |
| 9 | Below LCST: 27° C. | 0.18448 |
| 10 | Above LCST: 42° C. | 0.27336 |
| 11 | Below LCST: 27° C. | 0.19576 |
| 12 | Above LCST: 42° C. | 0.27027 |

The results show that the most mechanically strong coatings were produced after microcapillaries were pretreated with UV light, and then the esterification reaction of carboxyl groups of AA (acrylic acid) with the hydroxyls on the capillary surface was used to bond AA monomers on the glass surface. The pretreatment was followed by the copolymerization and grafting reactions. The experiments shows that such coatings can withstand delamination from the wall up to pressure drops of about 1 bar as well as periodic temperature variation across the lower critical solution temperature (LCST) in water resulting in coating swelling/shrinkage. Thermo-responsive flow regulation by periodic temperature variation across LCST demonstrated significant periodic variations of volumetric flow rate of the order of 50%.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A stimuli-responsive low solubility copolymer hydrogel composition comprising polymerized N-isopropylacrylamide and water insoluble monomer or oligomer repeating units, wherein said repeating units are randomly arranged within a polymer backbone of the copolymer hydrogel.

2. The copolymer hydrogel of claim 1, wherein said water insoluble monomer or oligomer repeating units comprise methyl methacrylate.

3. The copolymer hydrogel of claim 2, further comprising pH responsive monomer or oligomer repeating units.

4. The copolymer hydrogel of claim 3, wherein said pH responsive monomer or oligomer repeating units comprise acrylic acid.

5. The copolymer hydrogel of claim 1, further comprising pH responsive monomer or oligomer repeating units.

6. The copolymer hydrogel of claim 5, wherein said pH responsive monomer or oligomer repeating units comprise carboxyl groups or amino groups.

7. The copolymer hydrogel of claim 6, wherein said pH responsive monomer or oligomer repeating units comprise acrylic acid.

8. The copolymer hydrogel of claim 6, wherein said pH responsive monomer or oligomer repeating units comprise poly(methyl methacrylate).

9. The copolymer hydrogel of claim 1, wherein said water insoluble monomer or oligomer repeating units comprise are produced from monomer or oligomer macromolecules that can be synthesized as the copolymer hydrogel with NIPAM by Free-Radical polymerization.

10. The copolymer hydrogel of claim 1, wherein said water insoluble monomer or oligomer repeating units comprise polyvinyl chloride.

11. The copolymer hydrogel of claim 1, wherein said water insoluble monomer or oligomer repeating units comprise polystyrene.

12. The copolymer hydrogel of claim 1, wherein said water insoluble monomer or oligomer repeating units comprise polyacrylonile.

13. The copolymer hydrogel of claim 1, formed as bulk material.

14. The copolymer hydrogel of claim 1, formed as a nanofiber mat.

15. The copolymer hydrogel of claim 1, embedded with a substance to be delivered in response to stimuli.

16. The copolymer hydrogel of claim 14, wherein the substance comprises a drug.

17. The copolymer hydrogel of claim 1, formed as a coating.

18. A method for synthesis of a stimuli-responsive low solubility copolymer hydrogel composition comprising polymerized N-isopropylacrylamide and water insoluble monomer or oligomer repeating units, the method comprising steps of:
solving precursors of N-isopropylacrylamide and precursors of the water insoluble monomer of oligomer in a liquid solvent to form a solution in a container,
adding an initiator to the solution;
bubbling gas through the solution and then sealing the container; and
stirring the solution while heating to a polymerization temperature and permitting polymerization to complete to form the copolymer hydrogel composition.

19. The method according to claim 18, further comprising steps of:
opening the container and evaporating the solvent;
adding the residual material gradually to hexane while stirring to precipitate the copolymer composition as sediment;
removing hexane with the residual monomers;
washing the copolymer sediment; and
drying the copolymer sediments to leave the copolymer hydrogel composition.

20. The method according to claim 18, wherein said step of solving further comprises solving a precursor of a pH sensitive monomer or oligomer with the precursors of N-isopropylacrylamide and precursors of the water insoluble monomer or oligomer.

21. The method according to claim 18, further comprising a step of electrospinning the copolymer hydrogel composition into a fiber mat.

22. The method according to claim 18, further comprising a step of coating the copolymer hydrogel composition onto a surface.

23. The method according to claim 22, further comprising a preliminary step of treating the surface for copolymer bonding.
